(19) United States
(12) Patent Application Publication
(10) Pub. No.: US 2019/0090755 A1
(43) Pub. Date: Mar. 28, 2019

(54) Pulse Wave Detector

(71) Applicant: Yamaha Corporation, Hamamatsu-shi (JP)

(72) Inventors: Yasuhiko SEKIMOTO, Hamamatsu-shi (JP); Norihiro KAWAGISHI, Hamamatsu-shi (JP); Yasuo NITTA, Hamamatsu-shi (JP)

(21) Appl. No.: 16/139,313

(22) Filed: Sep. 24, 2018

(30) Foreign Application Priority Data
Sep. 26, 2017 (JP) 2017-184789

(51) Int. Cl. A61B 5/022; A61B 5/00

(52) U.S. Cl. CPC A61B 5/022; A61B 5/02427; A61B 5/6843; A61B 5/6824

(57) ABSTRACT

A pulse wave detector includes: a base portion; a pulse wave sensor that is provided on a side of one surface of the base portion; and a pressure adjusting portion that adjusts pressure for pressing the pulse wave sensor in a direction in which the one surface of the base portion faces.

### CROSS-REFERENCE TO RELATED APPLICATION

Priority is claimed on Japanese Patent Application No. 2017-184789, filed Sep. 26, 2017, the content of which is incorporated herein by reference.

### BACKGROUND OF THE INVENTION

### Field of the Invention

The present invention relates to a pulse wave detector.

### Description of Related Art

There has conventionally been provided a pulse wave detector that presses a pulse wave sensor consisting of a piezoelectric sensor against a living body part (skin) through which passes an artery, such as a radial artery at the wrist, and detects a pulse wave (pulse vibration) of the living body by the pulse wave sensor (refer to PCT International Publication No. WO 2017/043259 and PCT International Publication No. WO 2017/043260, for example). A pulse wave of a living body detected by a pulse wave sensor is used for measuring biological information such as pulse and blood pressure.

### SUMMARY OF THE INVENTION

In order to continuously detect a pulse wave of a living body in daily life with a pulse wave detector, it is necessary to press (squeeze) the pulse wave sensor against the user's skin over a prolonged period a long time. However, strongly pressing a pulse wave sensor against skin may cause the user to feel discomfort. In addition, if the pressure for pressing the pulse wave sensor against the skin is excessively weak or strong, the pulse wave may not be detected correctly in some cases.

The present invention has been achieved in view of the above circumstances. An exemplary object of the present invention is to provide a pulse wave detector capable of correctly detecting a pulse wave of a user over a prolonged period of time while alleviating discomfort of a user.

A pulse wave detector according to an aspect of the present invention includes: a base portion; a pulse wave sensor that is provided on a side of one surface of the base portion; and a pressure adjusting portion that adjusts pressure for pressing the pulse wave sensor in a direction in which the one surface of the base portion faces.

According to the present invention, it is possible to correctly detect over a prolonged period of time the pulse wave of the user wearing the pulse wave detector while reducing discomfort of the user.

### BRIEF DESCRIPTION OF THE DRAWINGS

FIG. 1 is a schematic cross-sectional view showing the pulse wave detector according to the first embodiment of the present invention.

FIG. 2 is a schematic cross-sectional view of the pulse wave detector of FIG. 1 as seen from the one surface side of the elastic membrane portion.

FIG. 3 is a schematic cross-sectional view showing a state in which the pulse wave detector of FIGS. 1 and 2 is worn on a wrist of a living body.

FIG. 4 is a schematic cross-sectional view showing a state in which the pulse wave detector according to the second embodiment of the present invention is worn on a wrist of a living body.

FIG. 5 is a plan view of the pulse wave sensor of FIG. 4, the pressure sensor and the gel as seen from the inner peripheral surface side of the band.

FIG. 6 is a schematic cross-sectional view showing a state in which the pulse wave detector according to the third embodiment of the present invention is worn on a wrist of a living body.

FIG. 7 is a schematic cross-sectional view showing a state in which the pulse wave detector according to the fourth embodiment of the present invention is worn on a wrist of a living body.

### DETAILED DESCRIPTION OF THE INVENTION

### First Embodiment

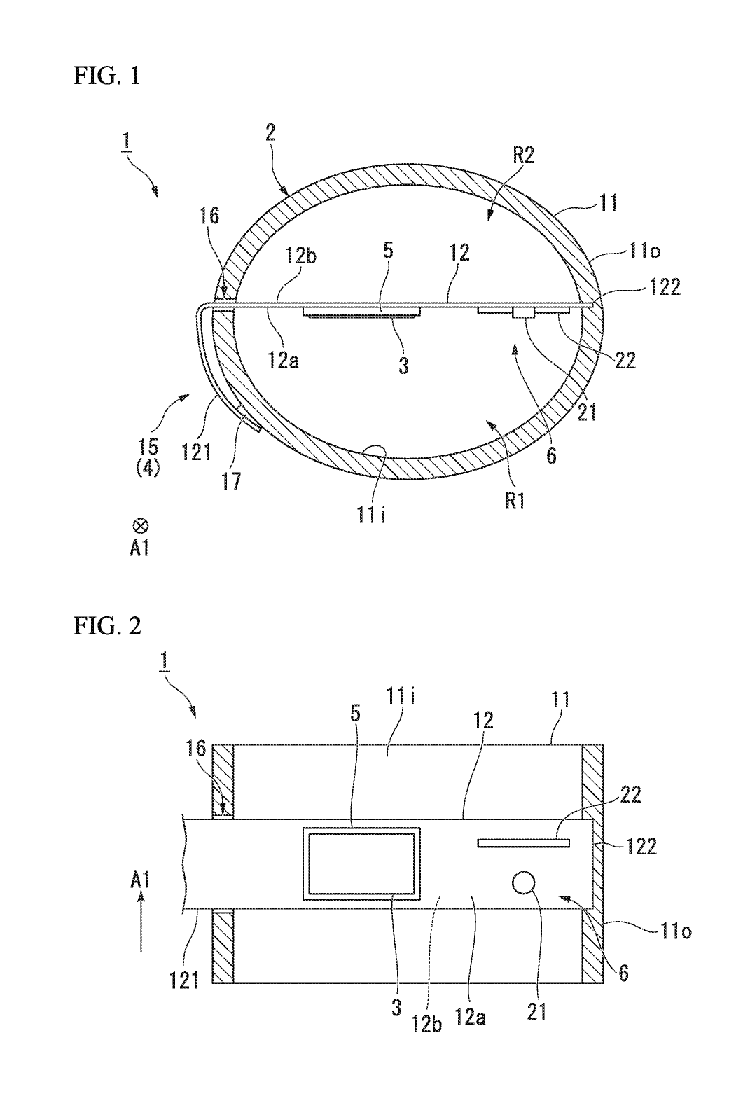

Hereinbelow, the first embodiment of the present invention will be described with reference to FIGS. 1 to 3.

As shown in FIG. 1, a pulse wave detector 1 according to the present embodiment includes a base portion 2, a pulse wave sensor 3, and a pressure adjusting portion 4.

The base portion 2 may be arbitrarily constituted provided the base portion 2 has at least a surface facing the skin side of a living body (hereinafter referred to as one surface). The base portion 2 of the present embodiment includes a ring portion 11 and an elastic membrane portion 12.

The ring portion 11 is formed in an annular shape. The region inside the ring portion 11 should have a size large enough to pass at least a part of a living body such as a wrist. The shape of the ring portion 11 as viewed from the axial direction (the A1 direction in the illustrated example) may be an elliptical shape as shown in the drawing, but may alternatively be any other shape such as a circle or a polygon, for example. The ring portion 11 should have a higher elastic modulus than at least the elastic membrane portion 12.

As shown in FIGS. 1 and 2, the elastic membrane portion 12 is formed in an elastically deformable belt shape. The elastic membrane portion 12 is provided so as to traverse the inside of the ring portion 11. Viewing the ring portion 11 from the axial direction, the elastic membrane portion 12 is provided so as to divide a region inside the ring portion 11 into two divided regions R1 and R2. The sizes of the two divided regions R1 and R2 may be equal to each other or may be different. In FIG. 1, the size of the first divided region R1 (the divided region on the lower side) is slightly larger than the size of the second divided region R2 (the divided region on the upper side).

The elastic membrane portion 12 is provided so that both surfaces (one surface 12a and the other surface 12b) of the elastic membrane portion 12 face the inner periphery 11i of the ring portion 11.

The dimension (width dimension) of the elastic membrane portion 12 in the axial direction of the ring portion 11 may for example be equal to or greater than the dimension (axial dimension dimension) of the ring portion 11, but in the present embodiment is smaller than the axial direction dimension of the ring portion 11. The elastic membrane portion 12 is located inside both ends of the ring portion 11 in the axial direction.

The pulse wave sensor 3 is a piezoelectric sensor that converts vibration (pressure) such as a pulse wave into a voltage. The shape of the pulse wave sensor 3 may be arbitrarily determined, but in the present embodiment is formed into a film shape or a sheet shape. The material used for the pulse wave sensor 3 may be an inorganic material such as lead zirconate titanate. In the present embodiment, the piezoelectric material used for the pulse wave sensor 3 is a flexible polymer material. The polymer material used for the pulse wave sensor 3 may be arbitrarily determined, with examples thereof including polyvinylidene fluoride (PVDF), polypropylene (PP) or the like. The pulse wave sensor 3 of the present embodiment is a porous electret including a large number of flat pores having small dimensions in the thickness direction thereof.

The pulse wave sensor 3 composed of the porous electret can correctly detect a pulse wave when the pressure for pressing the pulse wave sensor 3 against the skin of a living body is within a predetermined range. The range of the pressure for pressing the pulse wave sensor 3 is, for example, larger than 0 mmHg and smaller than 10 mmHg, and more preferably equal to or greater than 1.25 mmHg and equal to or less than 5 mmHg.

The pulse wave sensor 3 is provided on one surface side of the base portion 2. One surface of the base portion 2 may be, for example, the inner circumference 11i of the ring portion 11. The one surface of the base portion 2 in this embodiment is the one surface 12a of the elastic membrane portion 12 facing the inner periphery 11i of the ring portion 11.

For example, the pulse wave sensor 3 may be provided directly on the one surface 12a of the elastic membrane portion 12 (base portion 2). In the pulse wave detector 1 of the present embodiment, the pulse wave sensor 3 and the gel 5 are provided in an overlapping manner on the one surface 12a of the elastic membrane portion 12. For example, the pulse wave sensor 3 and the gel 5 may overlap in this order on the one surface 12a of the elastic membrane portion 12. In the present embodiment, the gel 5 and the pulse wave sensor 3 overlap in this order on the one surface 12a of the elastic membrane portion 12.

The gel 5 is formed in a sheet shape having viscoelasticity. The gel 5 uniformly transmits the pressing force and the pulse wave (vibration) to the entire surface of the pulse wave sensor 3 in a state where the pulse wave sensor 3 is pressed against the skin of a living body. That is, the gel 5 prevents the pressing force and the pulse wave (vibration) against the pulse wave sensor 3 in the thickness direction of the pulse wave sensor 3 from acting on only a partial region of the surface of the pulse wave sensor 3.

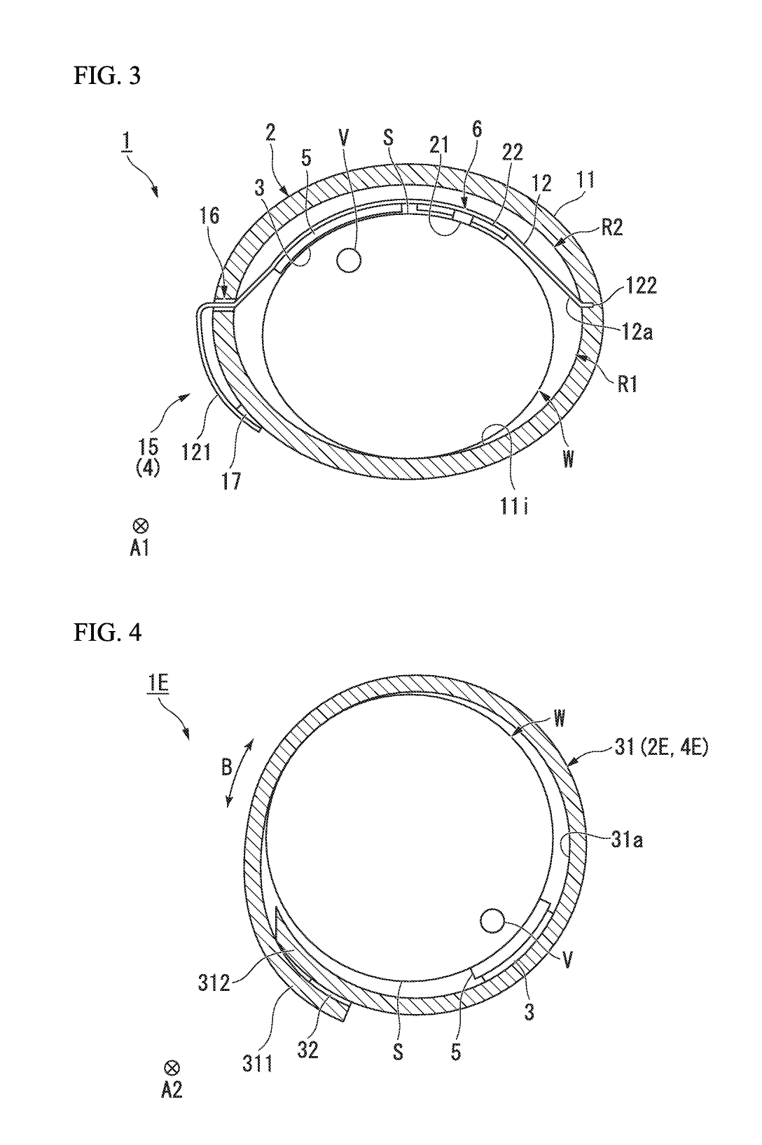

As shown in FIG. 3, for example, the pulse wave detector 1 according to the present embodiment having the ring portion 11 and the elastic membrane portion 12 can be worn on a wrist W of a human body by passing the wrist W through the region between the one surface 12a of the elastic membrane portion 12 and the inner periphery 11i of the ring portion 11 (that is, the first divided region R1). In this state, the pulse wave sensor 3 is pressed against the skin S of the living body by the elastic force of the elastic membrane portion 12. Thereby, the pulse wave sensor 3 can detect the pulse wave in the artery V (for example, the radial artery) passing through the inside of the wrist W.

As shown in FIGS. 1 and 2, the pressure adjusting portion 4 adjusts the pressure for pressing the pulse wave sensor 3 in the direction in which the one surface 12a of the base portion 2 faces. The pressing force exerted on the pulse wave sensor 3 by the pressure adjusting portion 4 may be in a range larger than 0 mmHg and smaller than 10 mmHg, for example. The pressure adjusting portion 4 in the present embodiment is a tension adjusting portion 15 for adjusting the tension of the elastic membrane portion 12 stretched inside the ring portion 11. The specific configuration of the tension adjusting portion 15 may be arbitrarily determined.

The tension adjusting portion 15 of the present embodiment includes a slit 16 formed in the ring portion 11 and a retaining portion 17 for retaining the elastic membrane portion 12 on the ring portion 11.

The slit 16 is formed penetrating the ring portion 11 in the radial direction thereof from the inside to the outside. The slit 16 plays the role of passing a first end portion 121 of the elastic membrane portion 12 in the longitudinal direction thereof from the inside to the outside of the ring portion 11. A second end portion 122 of the elastic membrane portion 12 in the longitudinal direction thereof is affixed to the inner periphery of the ring portion 11.

The retaining portion 17 retains the first end portion 121 of the elastic membrane portion 12 extending outside the ring portion 11 through the slit 16 at the outer periphery 11o of the ring portion 11. The retaining portion 17 is configured so as to retain the first end portion 121 of the elastic membrane portion 12 at an arbitrary position in the circumferential direction of the outer periphery 110 of the ring portion 11. The retaining portion 17 may be configured so as to detachably retain at least the first end portion 121 of the elastic membrane portion 12 at the outer periphery 11o of the ring portion 11, and may, for example, be a surface fastener, a snap button or the like.

By changing the position at which the elastic membrane portion 12 is retained on the outer periphery 11o of the ring portion 11 with the tension adjusting portion 15 of the present embodiment, the tension of the elastic membrane portion 12 inside the ring portion 11 can be adjusted. For example, when the first end portion 121 of the elastic membrane portion 12 is retained at the outer periphery 11o of the ring portion 11 by the retaining portion 17 at a position further away from the slit 16 of the ring portion 11, the tension of the elastic membrane portion 12 is increased. In this state, when the pulse wave detector 1 is worn on the wrist as illustrated in FIG. 3, the elastic force of the elastic membrane portion 12 pressing the pulse wave sensor 3 against the skin S of the living body increases. That is, by adjusting the tension of the elastic membrane portion 12, it is possible to change the pressing force acting on the pulse wave sensor 3.

The tension adjustment of the elastic membrane portion 12 may be performed in a state in which the pulse wave detector 1 is worn on the wrist W or may be performed in a state in which the pulse wave detector 1 is removed from the wrist W.

As shown in FIGS. 1 and 2, the pulse wave detector 1 of the present embodiment further includes a pressing force detecting portion 6. The pressing force detecting portion 6 detects the pressing force exerted on the pulse wave sensor 3 by the pressure adjusting portion 4.

The pressing force detecting portion 6 may be constituted by, for example, the pulse wave sensor 3. In this case, the pressing force of the pulse wave sensor 3 may be found on the basis of the differential between the output value of the pulse wave sensor 3 in the state of a pressing force not being exerted on the pulse wave sensor 3 by the pressure adjusting portion 4 and the output value of the pulse wave sensor 3 in the state of a pressing force being exerted on the pulse wave sensor 3 by the pressure adjusting portion 4.

The pressing force detecting portion 6 of the present embodiment is configured by a pressure sensor 21 and a tension sensor 22 provided on the one surface 12a of the elastic membrane portion 12 (base portion 2). The pressure sensor 21 detects the pressing force of the pulse wave sensor 3 by being pressed against the skin of a living body, similarly to the pulse wave sensor 3. The tension sensor 22 detects the tension of the elastic membrane portion 12 stretched inside the ring portion 11.

The tension of the elastic membrane portion 12 is a parameter corresponding to the pressing force exerted on the pulse wave sensor 3 as described above. For example, the tension sensor 22 may be provided on the other surface 12b of the elastic membrane portion 12. Although the pulse wave detector 1 illustrated in FIGS. 1 and 2 includes both the pressure sensor 21 and the tension sensor 22, the pulse wave detector 1 may include only one of them for example.

In FIG. 2, the pressure sensor 21 and the tension sensor 22 are arranged so as to be adjacent to the pulse wave sensor 3 in the longitudinal direction of the elastic membrane portion 12 (the direction orthogonal to the axial direction of the ring portion 11), but the arrangement is not limited to thereto. For example, the pressure sensor 21 and the tension sensor 22 may also be arranged so as to be adjacent to the pulse wave sensor 3 in the axial direction of the ring portion 11.

As described above, according to the pulse wave detector 1 of the present embodiment, by adjusting the pressure for pressing the pulse wave sensor 3 by the pressure adjusting portion 4, it is possible to prevent the pulse wave sensor 3 from being pressed against the skin S of the user (living body) with excessive pressure. In addition, it is also possible to prevent the pressure for pressing the pulse wave sensor 3 against the skin S from becoming excessively weak. As a result, it is possible to detect a pulse wave of a user correctly over a prolonged period of time while alleviating discomfort of the user.

Further, in the pulse wave detector 1 of the present embodiment, the pulse wave sensor 3 is a porous electret. Therefore, even if the pressure for pressing the pulse wave sensor 3 against the user's skin is small (for example, less than 10 mmHg), a pulse wave of the user can be correctly detected. The pressure for pressing the pulse wave sensor 3 that does not lead to discomfort of the user is, for example, 30 mmHg or less. For this reason, it is possible to correctly detect a pulse wave of a user over a prolonged time while further alleviating discomfort experienced by the user.

The pulse wave detector 1 of the present embodiment includes the pressing force detecting portion 6 that detects the pressing force exerted on the pulse wave sensor 3 by the pressure adjusting portion 4. For this reason, on the basis of the pressing force detected by the pressing force detecting portion 6, it is possible to appropriately adjust the pressure for pressing the pulse wave sensor 3 against the skin S by the pressure adjusting portion 4.

In the pulse wave detector 1 of the present embodiment, the base portion 2 is constituted by the ring portion 11 and the elastic membrane portion 12. For this reason, simply by passing a part of the user such as a wrist W through the inside of the ring portion 11, the pulse wave detector 1 can be easily mounted on the user's wrist W.

In the pulse wave detector 1 of the present embodiment, the portion of the elastic membrane portion 12 to which the pulse wave sensor 3 is attached is positioned inside the ring portion 11. Therefore, in a state in which the pulse wave detector 1 is attached to a user's wrist W or the like, it is possible to effectively inhibit the pulse wave sensor 3 from being subjected to an unexpected external forces as a result of being pressed against the user's skin S by the user's fingers, walls, or the like. Accordingly, it is possible to correctly detect a pulse wave of the user by suppressing the application of unexpected external forces to the pulse wave sensor 3.

In addition, in the pulse wave detector 1 of the present embodiment, the pressure adjusting portion 4 is constituted by the tension adjusting portion 15 that adjusts the tension of the elastic membrane portion 12. The tension adjusting portion 15 is constituted by the slit 16 formed in the ring portion 11 for passing therethrough the first end portion 121 of the elastic membrane portion 12, and the retaining portion 17 that retains on the ring portion 11 the first end portion 121 of the elastic membrane portion 12 extending to the outside of the ring portion 11. Therefore, the tension of the elastic membrane portion 12 can be easily changed just by changing the position on the outer periphery 11o of the ring portion 11 (the position in the circumferential direction of the ring portion 11) at which the first end portion 121 of the elastic membrane portion 12 is retained by the retaining portion 17.

The first end portion 121 of the elastic membrane portion 12 can be held by the retaining portion 17 at the position on the outer periphery 11o of the ring portion 11 at which the pressure for pressing the pulse wave sensor 3 against the user's skin is appropriate. That is, the tension of the elastic membrane portion 12 can be kept constant even if the pulse wave detector 1 is not mounted. This eliminates the need to adjust the pressure for pressing the pulse wave sensor 3 against the skin S of the user each time the pulse wave detector 1 is attached to the user's wrist W or the like. In other words, it is possible to enhance the handling of the pulse wave detector 1.

### Second Embodiment

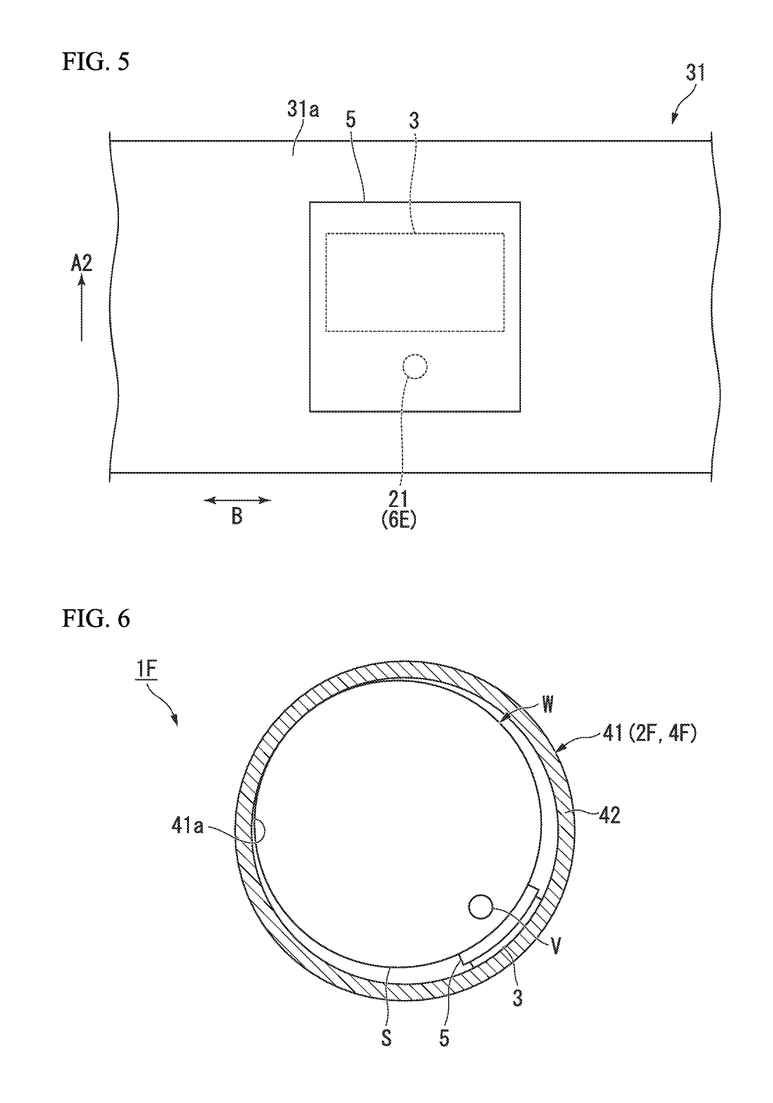

Next, a second embodiment of the present invention will be described with reference to FIGS. 4 and 5. Constituent elements of the present embodiment that are the same as those of the first embodiment are denoted by the same reference numerals, with descriptions thereof being omitted.

As shown in FIGS. 4 and 5, a pulse wave detector 1E according to the present embodiment includes a base portion 2E, the pulse wave sensor 3, and a pressure adjusting portion 4E similarly to the first embodiment.

The base portion 2E of the present embodiment is a band 31 that is formed in a belt shape having flexibility and intended to be wrapped around a portion of a living body such as a wrist W. The band 31 may be formed so as to be elastically deformable in, for example, the longitudinal direction thereof (direction B in FIGS. 4 and 5).

The band 31 has a retaining portion 32 for mutually retaining a first end portion 311 and a second end portion 312 of the band 31 in the longitudinal direction thereof in the state of being wrapped in an annular shape. The retaining portion 32 is configured so that the first end portion 311 of the band 31 can be retained at an arbitrary position in the circumferential direction of the second end portion 312. Thereby, the annular size of the band 31 can be changed. The specific structure of the retaining portion 32 may be a surface fastener, a snap button or the like similarly to the retaining portion 17 of the first embodiment (refer to FIGS. 1 and 3).

The pulse wave sensor 3 of the present embodiment is provided on one surface side of the band 31 (base portion 2E). The one surface of the band 31 is an inner peripheral surface 31a of the band 31 wrapped in an annular shape. The pulse wave sensor 3 may be disposed at an arbitrary position in the longitudinal direction of the band 31 within a range that does not interfere with the aforementioned retaining portion 32.

In the present embodiment, as in the case of the first embodiment, the pulse wave sensor 3 and the gel 5 are provided in an overlapping manner on the inner peripheral surface 31a of the band 31. The order in which the pulse wave sensor 3 and the gel 5 overlap may be arbitrarily determined. In the present embodiment, the pulse wave sensor 3 and the gel 5 overlap in this order on the inner peripheral surface 31a of the band 31.

The pressure adjusting portion 4E of the present embodiment is constituted by the band 31 having the retaining portion 32. In the present embodiment, by changing the relative positions of the first end portion 311 and the second end portion 312 of the band 31 in the circumferential direction of the annular band that is made annular by being wrapped around a part of a living body such as a wrist W or the like, it is possible to adjust the pressure for pressing the pulse wave sensor 3 against the skin S of a living body (in the direction in which the inner peripheral surface 31a of the band 31 faces).

As in the first embodiment, the pulse wave detector 1E of the present embodiment includes a pressing force detecting portion 6E that detects the pressing force exerted on the pulse wave sensor 3 by the pressure adjusting portion 4E. The pressing force detecting portion 6E of the present embodiment may be constituted by, for example, the pulse wave sensor 3, but in the illustrated example is constituted by a pressure sensor 21 similar to that of the first embodiment provided on the inner peripheral surface 31a of the band 31.

The pressure sensor 21 may for example be disposed so as to be adjacent to the pulse wave sensor 3 in the longitudinal direction of the band 31. In the present embodiment, the pressure sensor 21 is arranged so as to be adjacent to the pulse wave sensor 3 in the width direction of the band 31 (direction A2 in FIGS. 4 and 5). The pressure sensor 21 may also be disposed so as to overlap the gel 5 similarly to the pulse wave sensor 3 as shown in FIG. 5, but may also not overlap the gel 5, for example.

The pulse wave detector 1E of the present embodiment exhibits the same effect as that of the first embodiment.

### Third Embodiment

Next, a third embodiment of the present invention will be described with reference to FIG. 6. Constituent elements of the present embodiment that are the same as those of the first embodiment are denoted by the same reference numerals, with descriptions thereof being omitted.

As shown in FIG. 6, similarly to the first embodiment, a pulse wave detector 1F of the present embodiment includes a base portion 2F, a pulse wave sensor 3, and a pressure adjusting portion 4F.

The base portion 2F of the present embodiment is a cuff 41 having a cavity 42 therein capable of being inflated and deflated. The cuff 41 may be formed in a cylindrical shape having flexibility as in the illustrated example, and may, similarly to example the band 31 of the second embodiment (see FIGS. 4 and 5), be formed in a belt shape having flexibility and configured to be wrappable around a part of a living body such as a wrist W.

The pulse wave sensor 3 of the present embodiment is provided on the inner peripheral surface 41a side of the cuff 41 (one surface side of the base portion 2F). In the present embodiment, as in the case of the first embodiment, the pulse wave sensor 3 and the gel 5 are provided in an overlapping manner on the inner peripheral surface 41a of the cuff 41. The order in which the pulse wave sensor 3 and the gel 5 overlap may be arbitrarily determined. In the present embodiment, the pulse wave sensor 3 and the gel 5 overlap in this order on the inner peripheral surface 41a of the cuff 41.

The pressure adjusting portion 4F of the present embodiment is constituted by the cuff 41. In the present embodiment, it is possible to adjust the pressure for pressing the pulse wave sensor 3 against the skin S of a living body (in the direction in which the inner peripheral surface 41a of the cuff 41 faces) by changing the air pressure in the cuff 41 by an air pressure adjusting portion such as an air pump (not illustrated). The air pressure adjusting portion may or may not be included in the configuration of the pressure adjusting portion 4F together with the cuff 41. That is, the air pressure adjusting portion may be included in the configuration of the pulse wave detector 1F, or may be configured separately from the pulse wave detector 1F.

The pulse wave detector 1F of the present embodiment may include a pressing force detecting portion similar to that of the first and second embodiments. The pressing force detecting portion may be constituted by the pulse wave sensor 3, or may be constituted by a pressure sensor 21 similar to that of the first and second embodiments (see FIG. 5). In the case where the pressing force detecting portion is the pressure sensor 21, the pressure sensor 21 should be provided on the inner peripheral surface 41a of the cuff 41.

The pulse wave detector 1F of the present embodiment exhibits the same effects as those of the first embodiment.

### Fourth Embodiment

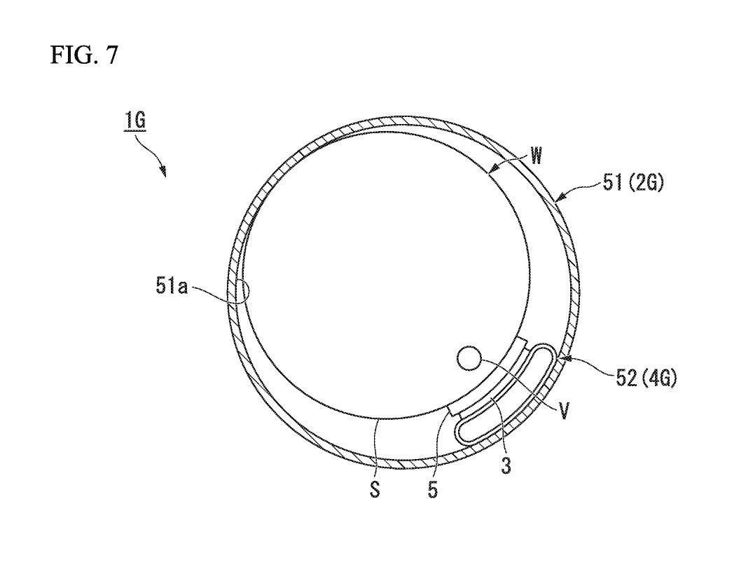

Next, a fourth embodiment of the present invention will be described with reference to FIG. 7. Constituent elements of the present embodiment that are the same as those of the first embodiment are denoted by the same reference numerals, with descriptions thereof being omitted.

As shown in FIG. 7, a pulse wave detector 1G of the present embodiment includes a base portion 2G, a pulse wave sensor 3, and a pressure adjusting portion 4G similarly to the first embodiment.

The base portion 2G may be, for example, the band 31 formed in a belt shape as in the second embodiment, but in the present embodiment is an annular band 51 having flexibility. The annular band 51 may for example be elastically deformable but is not limited thereto.

The pulse wave sensor 3 of the present embodiment is provided on an inner peripheral surface 51a side of the band 51 (one surface side of the base portion 2G). In the present embodiment, as in the case of the first embodiment, the pulse wave sensor 3 and the gel 5 are provided in an overlapping manner on the inner peripheral surface 51a of the band 51. The order in which the pulse wave sensor 3 and the gel 5 overlap may be arbitrarily determined. In the present embodiment, the pulse wave sensor 3 and the gel 5 overlap in this order on the inner peripheral surface 51a of the band 51.

The pressure adjusting unit 4G of the present embodiment is an air bladder 52 provided between the inner peripheral surface 51a of the band 51 and the pulse wave sensor 3. The air bladder 52 is constituted to be able to take in and let out air. In the present embodiment, by changing the air pressure in the air bladder 52 by an air pressure adjusting portion (not shown) such as an air pump, it is possible to adjust the pressure for pressing the pulse wave sensor 3 against the skin S of a living body (in the direction in which the inner circumferential surface 51a of the band 51 faces).

In the inner peripheral surface 51a, the air bladder 52 is provided in the region where the pulse wave sensor 3 and the gel 5 are disposed, or in that region and an adjacent region. That is, the air bladder 52 is provided only in a part of the inner peripheral surface 51a of the band 51 in the circumferential direction thereof.

The aforementioned air pressure adjusting portion may or may not be included in the constitution of the pressure adjusting portion 4F together with the air bladder 52. That is, the air pressure adjusting portion may be included in the constitution of the pulse wave detector 1G, or may be configured separately from the pulse wave detector 1G.

The pulse wave detector 1G of the present embodiment may include a pressing force detecting portion similar to that of the first and second embodiments. The pressing force detecting portion may be constituted by the pulse wave sensor 3, or may be constituted by the pressure sensor 21 similar to that of the first and second embodiments (see FIG. 5). In the case where the pressing force detecting portion is the pressure sensor 21, the pressure sensor 21 should be provided so as to sandwich the air bladder 52 with the inner peripheral surface 51a of the band 51.

The pulse wave detecting device 1G of the present embodiment exhibits the same effect as the first embodiment.

According to the pulse wave detector 1G of the present embodiment, the pressure adjusting portion 4G is constituted by the air bladder 52 provided between the band 51 (base portion 2G) and the pulse wave sensor 3. For this reason, the air bladder 52 can reduce the volume of air to be filled, as compared with the cuff 41 (see FIG. 6) exemplified in the third embodiment. Therefore, it is possible to perform pressure adjustment in a short time when the pulse wave detector 1G is worn on the wrist W of the user.

The air bladder 52 of the present embodiment may be applied to for example the constitution of the first embodiment. The air bladder 52 may be provided between, for example, the elastic membrane portion 12 and the pulse wave sensor 3. In this case, both ends of the elastic membrane portion 12 in the longitudinal direction may be affixed to, for example, the inner periphery of the ring portion 11.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

In the pulse wave detector of the present invention, the base portion is not limited to a configuration that is wrapped around a part of a living body such as a wrist. For example, by having an adhesive layer on the one surface of the base portion, the pulse wave detector may be constituted to be adhered to the skin of a living body. In this case, the pressure adjusting portion may be configured separately from the base portion, in the manner of the air bladder exemplified in the fourth embodiment.

The pulse wave detector of the present invention may include a plurality of (for example, two) pulse wave sensors. In this case, the plurality of pulse wave sensors may be mutually arranged at an interval on the one surface side of the base portion. With such a configuration, by detecting a pulse wave with a plurality of pulse wave sensors, for example, the propagation time of a pulse wave can be measured.

What is claimed is:

1. A pulse wave detector comprising:
a base portion;
a pulse wave sensor that is provided on a side of one surface of the base portion; and
a pressure adjusting portion that adjusts pressure for pressing the pulse wave sensor in a direction in which the one surface of the base portion faces.

2. The pulse wave detector according to claim 1, wherein pressing force exerted on the pulse wave sensor by the pressure adjusting portion is greater than 0 mmHg and less than 10 mmHg.

3. The pulse wave detector according to claim 1, wherein the pulse wave sensor comprises a porous electret.

4. The pulse wave detector according to claim 1, further comprising:
a pressing force detecting portion that detects pressing force exerted on the pulse wave sensor by the pressure adjusting portion.

5. The pulse wave detector according to claim 1,
wherein the base portion comprises: a ring portion having an annular shape; and an elastic membrane portion having a belt shape, the elastic membrane portion being elastically deformable, the elastic membrane portion traversing inside of the ring portion,
the pulse wave sensor is provided on a side of one surface of the elastic membrane portion, the elastic membrane portion being positioned in the inside of the ring portion, and
the pressure adjusting portion comprises a tension adjusting portion that adjusts tension of the elastic membrane portion.

6. The pulse wave detector according to claim 5,
wherein the elastic membrane portion comprises a first end portion that extends in a longitudinal direction of the elastic membrane portion,
the tension adjusting portion comprises: a slit that is provided in the ring portion; and a retaining portion that retains the first end portion of the elastic membrane portion to an outer periphery of the ring portion, and
the first end portion of the elastic membrane portion passes through the slit and extends from the inside of the ring portion to outside of the ring portion.

7. The pulse wave detector according to claim 1, wherein the pressure adjusting portion comprises an air bladder provided between the base portion and the pulse wave sensor
